(19) United States
(12) Patent Application Publication
(10) Pub. No.: US 2017/0160275 A1
(43) Pub. Date: Jun. 8, 2017

(54) BLOOD-BASED LATERAL-FLOW DIPSTICK ASSAY FOR DETECTION OF ENTERIC FEVER

(71) Applicants: FIRDAUSI QADRI, DHAKA (BD); JAKIA AMIN, DHAKA (BD)

(72) Inventors: FIRDAUSI QADRI, DHAKA (BD); JAKIA AMIN, DHAKA (BD)

(21) Appl. No.: 15/368,545

(22) Filed: Dec. 2, 2016

Related U.S. Application Data
(60) Provisional application No. 62/262,890, filed on Dec. 3, 2015.

(51) Int. Cl. G01N 33/558; G01N 33/553; G01N 33/53; G01N 33/543

(52) U.S. Cl. CPC G01N 33/558; G01N 33/54346; G01N 33/553; G01N 33/5306; G01N 2469/20

(57) ABSTRACT

The blood-based lateral-flow dipstick assay for detection of enteric fever is an immunochromatographic dipstick assay based on detection of Salmonella enterica serovar Typhi (S. Typhi) LPS-specific IgG in lymphocyte culture secretion. The dipstick has a test line of an S. Typhi strain antigen extending across a chromatographic stationary phase disposed on a backing, A conjugate pad of glass fibers is attached to the backing so that it overlaps the stationary phase. The conjugate pad is soaked in a first mammal's anti-human antibody (e.g., goat anti-human IgG) conjugated with a color indicator (e.g., colloidal gold nanoparticles exhibiting a wine red color). When dipped in a lymphocyte culture of a human enteric fever victim, the test line turns to the color of the color indicator. A control line of anti-first mammal antibody across the stationary phase changes to the color of the indicator so long as the conjugated anti-human antibody remains viable.

### CROSS-REFERENCE TO RELATED APPLICATION

This application claims the benefit of U.S. Provisional Patent Application Ser. No. 62/262,890, filed Dec. 3, 2015.

### BACKGROUND OF THE INVENTION

1. Field of the Invention

The present invention relates to assays for detecting and quantifying the content of an analyte in a sample, and particularly to a blood-based lateral-flow dipstick assay for detection of enteric fever.

2. Description of the Related Art

Enteric fever remains an important public health concern in many developing countries. There is a very real need for a low-tech, reliable and affordable diagnostic test that shows high sensitivity and specificity. Enteric fever can be the result of typhoid and paratyphoid fever, and is caused by infection with Salmonella enterica serover Typhi (S. Typhi) or Salmonella enterica serover Paratyphi (S. Paratyphi). Approximately 22 million cases of typhoid fever and 6 million cases of paratyphoid fever occur annually, resulting in over 100,000 deaths globally each year. The occurrence of enteric fever is mainly associated with a lack of proper sanitation and fecal contamination of water and food. Rapid accurate diagnosis followed by early treatment with suitable antibiotics can reduce the rate of morbidity and mortality due to enteric fever. As the clinical features of enteric fever are non-specific and overlap with other bacterial and viral febrile illnesses, rapid and accurate diagnosis remains a challenge, particularly in resource-poor settings,

Although microbiologic culturing of bone marrow culture is considered a “gold standard” for diagnosing individuals with enteric fever, it is clinically impractical due to its invasive nature. Therefore, microbiologic culturing of peripheral blood culture is often used as an alternative in regions with laboratory capacity. Unfortunately, blood culture shows poor sensitivity, ranging from 30%-70%, depending on different factors including blood volume and prior antibiotic treatment, may take up to two to seven days to confirm the diagnosis, and requires a well-equipped laboratory and expertise for microbiologic confirmation. The Widal test is the most widely used serological test for diagnosing individuals with enteric fever, but lacks specificity, especially in enteric fever endemic areas. Additional serologic tests include the TyphiDot, that detects IgM and IgG antibodies in peripheral blood to a 50 kDa outer membrane protein of S. Typhi, and the Tubex assay, that detects IgM responses in blood to S. Typhi O9 lipopolysaccharide. These assays have been associated with sensitivities and specificities of 56-95%, and specificities of 31-97% in field tests.

Molecular-based methods, including nucleic acid amplification tests, have been hampered in field tests by the low organism load and the presence of inhibitors in peripheral blood, along with reagent and equipment expense, and a lack of technical expertise, although such assays may have higher sensitivity than blood culture. The ELISA-based test for enteric fever is based on detection of antibodies secreted ex vivo by activated lymphocytes recovered from the peripheral circulation during acute infection. These lymphocytes have been stimulated by the recent infection, and require no ex vivo stimulation. Removing the plasma component of blood limits the confounding influence of pre-existing circulating antibodies that reflect prior exposure. These circulating antibodies can affect assay-specificity and have markedly limited the utility of plasma antibody-based assays in areas of the world endemic for enteric fever and salmonellosis. Thus, a blood-based lateral-flow dipstick assay for detection of enteric fever solving the aforementioned problems is desired.

### SUMMARY OF THE INVENTION

The blood-based lateral-flow dipstick assay for detection of enteric fever is a test strip having a backing with an elongated chromatographic stationary phase layer disposed thereon, as in a conventional test strip-type assay. A test line of Salmonella enterica serovar Typhi (S. Typhi) antigen extends transversely across the elongated chromatographic stationary phase layer. The S. Typhi antigen may be an antigen derived from S. Typhi strain Ty21a or an antigen derived from S. Typhi wild-type strain ST-004.

As in a conventional test strip-type assay, a color indicator in the visible light spectrum is further provided, along with a conjugate pad of absorbent fibers attached to the backing. Preferably, the color indicator is formed from colloidal gold nanoparticles exhibiting the color red in the visible light spectrum.

The conjugate pad has a first mammal's anti-human antibody adsorbed thereon, where the first mammal's anti-human antibody is conjugated to the color indicator. The conjugate pad overlaps the elongated chromatographic stationary phase layer. Additionally, a control line of anti-first mammal IgG antibody extends transversely across the elongated chromatographic stationary phase layer in a spaced apart manner; i.e., the test line is spaced apart from the control line.

The first mammal's anti-human antibody may be goat anti-human IgG, with the anti-first mammal IgG antibody being rabbit anti-goat IgG. Alternatively, the first mammal's anti-human antibody may be goat anti-human IgA, with the anti-first mammal IgG antibody being rabbit anti-goat IgG.

These and other features of the present invention will become readily apparent upon further review of the following specification and drawings.

### BRIEF DESCRIPTION OF THE DRAWINGS

FIG. 1 is a side view in section of a blood-based lateral-flow dipstick assay for detection of enteric fever according to the present invention.

FIG. 2 is a graph showing results of a dynamic light scattering (DLS) spectrum analysis on gold nanoparticles used as a color indicator in the blood-based lateral-flow dipstick assay for detection of enteric fever.

FIG. 3A is a graph showing optical density (OD) results over a range of pH values for optimization of the pH for conjugation of the gold nanoparticles of FIG. 2 with anti-human IgG antibody.

FIG. 3B is a graph showing optical density (OD) results over a range of pH values for optimization of the pH for conjugation of the gold nanoparticles of FIG. 2 with anti-human IgA antibody.

FIG. 4A is a graph showing optical density (OD) results over a range of anti-human IgG antibody quantities per volume of colloidal gold to determine a minimum amount of anti-human IgG antibody for conjugation with the gold nanoparticles of FIG. 2.

FIG. 4B is a graph showing optical density (OD) results over a range of anti-human IgA antibody quantities per volume of colloidal gold to determine a minimum amount of anti-human IgA antibody for conjugation with the gold nanoparticles of FIG. 2.

FIG. 5 is a plot illustrating S. Typhi LPS specific IgG responses in a lymphocyte culture supernatant prepared from differing patient groups.

Similar reference characters denote corresponding features consistently throughout the attached drawings.

### DETAILED DESCRIPTION OF THE PREFERRED EMBODIMENTS

The blood-based lateral-flow dipstick assay for detection of enteric fever 10 is a test strip having a backing 12 with an elongated chromatographic stationary phase layer 24 disposed thereon, as in a conventional test strip-type assay. The sample to be tested is applied to a sample pad 14 and is drawn through the dipstick by natural diffusion and with the aid of an adsorbent sink pad 16, which is provided on the opposite end of the dipstick. A test line 18 of Salmonella enterica serovar Typhi (S. Typhi) antigen extends transversely across the elongated chromatographic stationary phase layer 24. The S. Typhi antigen may be an antigen derived from S. Typhi strain Ty21a or an antigen derived from S. Typhi wild-type strain ST-004.

As in a conventional test strip-type assay, a color indicator in the visible light spectrum is further provided, along with a conjugate pad 22 of absorbent fibers, such as glass fibers or the like, attached to the backing 12. Preferably, the color indicator is formed from colloidal gold nanoparticles exhibiting the color red. The chromatographic stationary phase layer 24 may be formed from any suitable type of dry hydrophilic wicking material, such as a nitrocellulose membrane or the like. The conjugate release pad 22 is in contact with sample pad 14 so that the two pads are in free-flow communication with each other and the sample flows through each pad in turn and through chromatographic stationary phase layer 24.

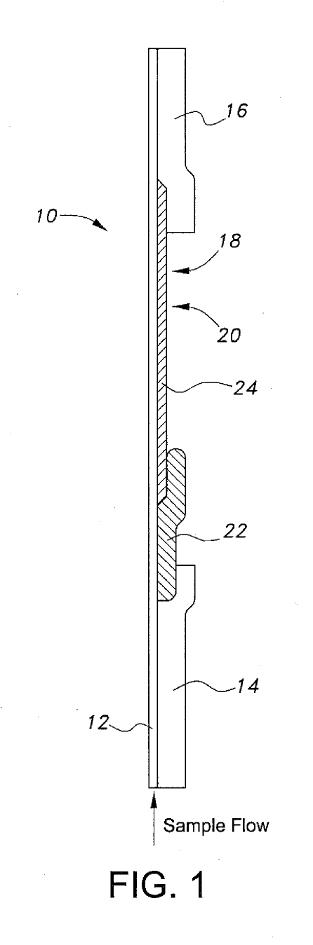

The conjugate pad 22 has a first mammal's anti-human antibody adsorbed thereon, where the first mammal's anti-human antibody is conjugated to the color indicator. The conjugate pad 22 overlaps the elongated chromatographic stationary phase layer 24, as shown in FIG. 1. Additionally, a control line 20 of anti-first mammal IgG antibody extends transversely across the elongated chromatographic stationary phase layer 24 in a spaced apart manner; i.e., the test line 18 is spaced apart from the control line 20.

The first mammal's anti-human antibody may be goat anti-human IgG, with the anti-first mammal IgG antibody being rabbit anti-goat IgG. Alternatively, the first mammal's anti-human antibody may be goat anti-human IgA, with the anti-first mammal IgG antibody being rabbit anti-goat IgG. The depth of color seen on test line 18 of the dipstick 10 indicates the quantity of S. Typhi contained in the sample and allows a diagnosis of enteric fever to be made when a positive result is obtained.

For purposes of the study to be described below, participants were enrolled who presented with clinically suspected enteric fever (n=142) which was defined as a systemic febrile illness with a body temperature of 38° C. or higher for a duration of three to seven days without any other obvious sources. An additional 35 study participants were enrolled who presented with a febrile illness confirmed to not be enteric fever, along with 28 healthy controls, all of whom resided in Dhaka, Bangladesh. A sample of venous blood was collected from each of the study participants. Below, Table 1 shows the general characteristics (median age and gender) of each group of study participants, and Table 2 shows the general characteristics for those suspected to have enteric fever.

TABLE 1
General Characteristics of Study Participants

| | | Patients with | |
|---|---|---|---|
| | Suspected | other febrile | |
| | Enteric | illness | Healthy controls |
| Features | fever (n = 140) | (n = 35) | (n = 28) |
| Median Age in years | 7 (3, 11) | 25 (23, 30) | 25 (25, 28) |
| (25^{th} and 75^{th} | | | |
| percentile) | | | |
| No. of male (%) | 70 (50) | 26 (74) | 18 (64) |
| No. of female (%) | 70 (50) | 9 (26) | 10 (36) |

TABLE 2
General Characteristics of Study Participants with Suspected Enteric Fever

| | | Blood | | Blood |
|---|---|---|---|---|
| | | culture | Blood | culture |
| | Blood | positive | culture | negative |
| | culture and | and | and | and |
| | Dipstick | Dipstick | Dipstick | Dipstick |
| Features | positive | negative | negative | positive |
| Sample size | 53 | 1 | 69 | 19 |
| Median Age in years | 6 (3, 12) | 45 | 7 (3, 12) | 7 (2, 11) |
| (25^{th} and 75^{th} percentile) | | | | |
| No. of male (%) | 18 (34) | 0 | 37 (54) | 12 (63) |
| No. of female (%) | 35 (66) | 1 | 32 (46) | 7 (37) |
| Duration of fever in | 5 (5, 6) | 5 | 4 (3, 5) | 5 (4, 6) |
| days | | | | |
| (25^{th} and 75^{th} percentile) | | | | |
| Prior use antibiotics (%) | 9 (17) | 0 | 5 (7) | 5 (26) |

In order to diagnose enteric fever using the collected blood samples, 3-5 mL samples of peripheral blood were used. A microbiological culture was performed using a BacT/Alert® automated system, manufactured by bioMérieux, Inc. of N.C., using subculturing positive bottles on MacConkey agar, blood agar, and chocolate agar plates, and identifying colonies using standard biochemical tests and reaction with Salmonella-specific antisera.

In order to prepare the coating antigen for the blood-based lateral-flow dipstick assay for detection of enteric fever 10, membrane preparation (MP) and lipopolysaccharide (LPS) were used as coating antigens prepared from the Ty21a vaccine strain and S. Typhi wild-type strain (ST-004), respectively. The bacterial strain was cultivated on horse blood agar plates, and bacteria were harvested in buffer (5 mM MgCl₂, 10 mM Tris; pH-8.0). The bacterial suspension was sonicated five times at 60% amplitude and centrifuged at 1400 g for ten minutes. The supernatant was then transferred to fresh tubes and centrifuged at 14900 g for 30 minutes. The pellet was dissolved in harvest buffer and the protein content was determined by the Bradford protein assay, manufactured by Bio-Rad Laboratories, Inc. of California. The LPS antigen was prepared from a wild-type clinical isolate of S. Typhi isolated from a patient, using a phenol-water extraction procedure, followed by enzyme treatment with proteinase K, DNase, and RNase, and ultracentrifugation.

Colloidal gold was prepared by mixing 0.01% HAuCl_{4 with} 0.024% sodium citrate in water for injection (WFI) and boiling until the solution became red wine in color. The colloidal gold was then filtered through a 0.2 μin filter. The pH of the gold solution was adjusted to 8.0 (optimum pH for conjugation), and a range of goat anti-human-IgG and goat anti-human-IgA was tested to conjugate 1 mL of colloidal gold, eventually selecting 12 μg and 16 μg, respectively, based on the data provided below. 20% BSA was used for blocking, and the conjugated antibody-gold was centrifuged at 10,000 rpm for 45 minutes at 4° C. The supernatant was discarded, and the pellet was re-suspended in 0.02M Tris buffer containing 1% BSA. This solution was passed through a 0.2 μm filter and used as the detection conjugates.

To assess stability of the conjugate and to define the optimum pH and minimum concentration of antibody required for conjugating the colloidal gold, an aggregation assay was used. Specifically, 10% NaCl was added to the gold-protein suspension, which was then incubated for 10 minutes, and then stability and polydispersity were assessed based on absorbance at 520 nm, 580 nm and 600 nm.

In order to make the blood-based lateral-flow dipstick assay for detection of enteric fever 10, the antigen and antibody were dispensed on the chromatographic stationary phase layer 24, which was fixed to backing layer 12, with the antigen and antibody forming the test and control lines 18, 20, respectively. A nitrocellulose membrane formed the chromatographic stationary phase layer 24. Dispensing of the test and control lines 18, 20 on the stationary phase layer 24 was performed using a HM3030 lateral flow dispenser, manufactured by Shanghai KinBio Tech Co., Ltd. of China. The nitrocellulose membrane 24 was then dried for 90 minutes.

The conjugate pad 22 was made by soaking glass fibers (obtained from Shanghai KinBio Tech Co., Ltd. of China) in the gold conjugate solution, followed by drying for two hours. The conjugate pad 22 was then pasted on the backing card layer 12 so as to overlap the nitrocellulose membrane 24, as shown in FIG. 1. The glass fiber sample pad 14 was placed at the bottom of the backing card layer 12 to overlap with the conjugate pad 22 in order to facilitate the flow of sample from a specimen vial to the strip, as further illustrated in FIG. 1. To accelerate migration of the samples through the strip 10, cellulose fiber was used to form the absorbent pad 16 pasted on the backing card, opposite the conjugate pad 22. All pads were cut to make the desired strip shape by using Guillotine cutter models CT300 and ZQ2000, both manufactured by Shanghai KinBio Tech Co., Ltd. of China.

To recover peripheral lymphocytes, erythrocytes present in 1.5-2 mL of venous blood were lysed using a lysis buffer (0.15 M ammonium chloride; 1 mM potassium bicarbonate and 0.01 mM disodium EDTA) at a 1:5 dilution, and the sample was mixed by gently inverting the tube 3-5 times. The tube was then kept at room temperature for 10 minutes and centrifuged at 953 g for five minutes at 20° C. The supernatant was decanted and the pellet was re-suspended in 150 μL of RPMI-1640 medium supplemented with 10% heat inactivated fetal bovine serum, 1% penicillin-streptomycin, 1% sodium pyruvate and 1% L-glutamine. The suspended cells were cultured in culture vials at 37° C. without 5% CO_{2 for} 48 hours. The culture suspension was then harvested and centrifuged at 11600 g at 20° C. for five minutes to collect the supernatant.

The resultant strip 10 contained two lines on the nitrocellulose membrane 24; one line was the test line 18 containing MP or LPS antigen, and the other line was the control line 20 containing rabbit anti-goat IgG. The conjugate pad 22 contained goat anti-human IgG or goat anti-human IgA conjugated to colloidal gold. 75 μL of the lymphocyte culture supernatant was diluted with Tris (0.02 M)-BSA (1%)-Tween (3%) at a 1:1 dilution in a microcentrifuge tube and the strip 10 was dipped into the tube for 15 minutes. The test line 18 and/or control line 20 would appear as a red color. Appearance of both the control line 20 and test line 18 indicated that the sample was positive for the test undertaken. Appearance of only the control line 20 but no test line 18 indicated a negative result for the test.

The strip results were compared against those obtained by using an anti-S. Typhi LPS ELISA format. For the comparison, microtiter plates were coated with 100 μL of LPS (2.5 μg/mL) and blocked with 1% BSA in PBS. To detect antigen-specific responses, 100 μL of lymphocyte culture supernatant (1:2 dilution in 0.1% BSA-PBS-Tween) was added to the coated plates, which were then incubated for 90 minutes at 37° C. After washing with 0.05% PBS-Tween, rabbit anti-human IgG (1:1000 dilution) was added, conjugated to horseradish peroxidase and incubated for 90 minutes at 37° C. The plates were developed with orthophenylene diamine in 0.1 M sodium citrate buffer and 0.1% hydrogen peroxide, after washing the plates with 0.05% PBS-Tween. The plates were read kinetically at 450 nm for five minutes at 19 second intervals. The maximal rate of optical density change was expressed as milli-optical density absorbance units per minute. All results were normalized by dividing with readings of in-house pooled convalescent-phase standard sera of blood culture-confirmed typhoid patients, multiplied by 100, and the results were expressed as ELISA units.

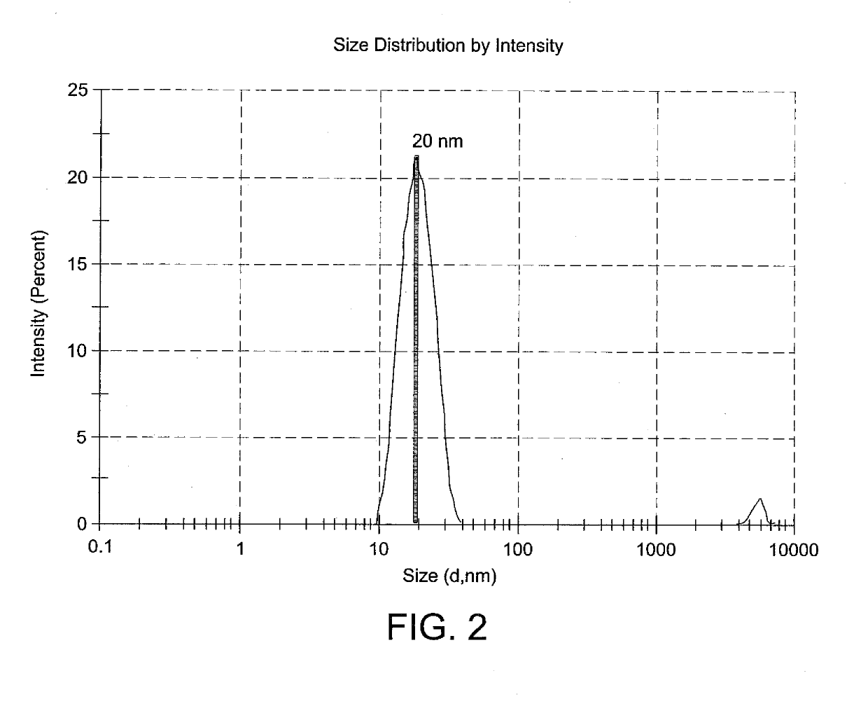

After preparing the colloidal gold, as described above, the size' of the gold nanoparticles was determined by differential light scatterings using a Zetasizer Nano ZS90 molecular/particle size and zeta potential analyzer, manufactured by Malvern Instruments, Ltd. of Worcester, England. The measurements were carried out at 25° C. with a count rate of 193.7 kcps at a scattering angle of 173°. The average diameter of the prepared gold nanoparticles was 20 nm, as determined from the dynamic light scattering (DLS) spectrum shown in FIG. 2.

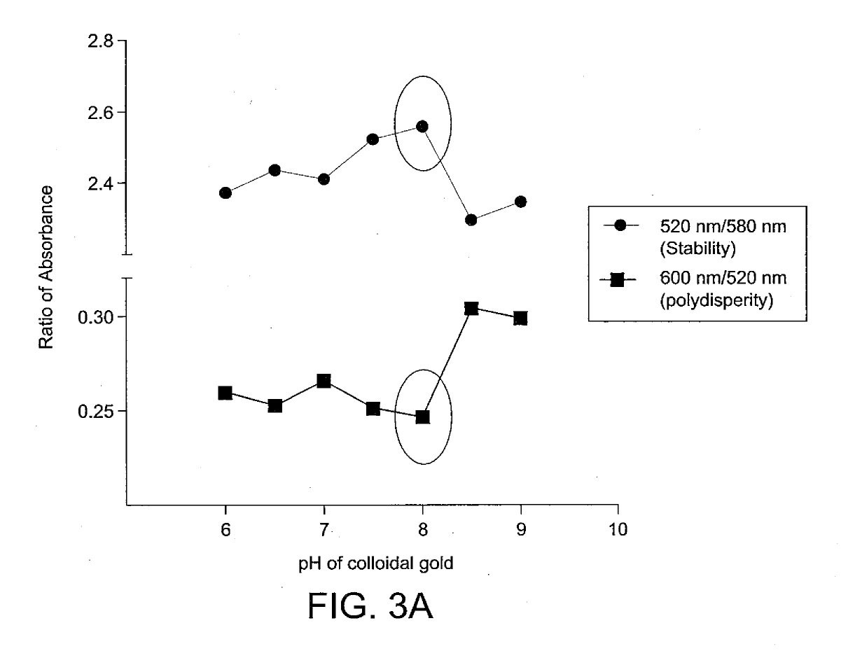

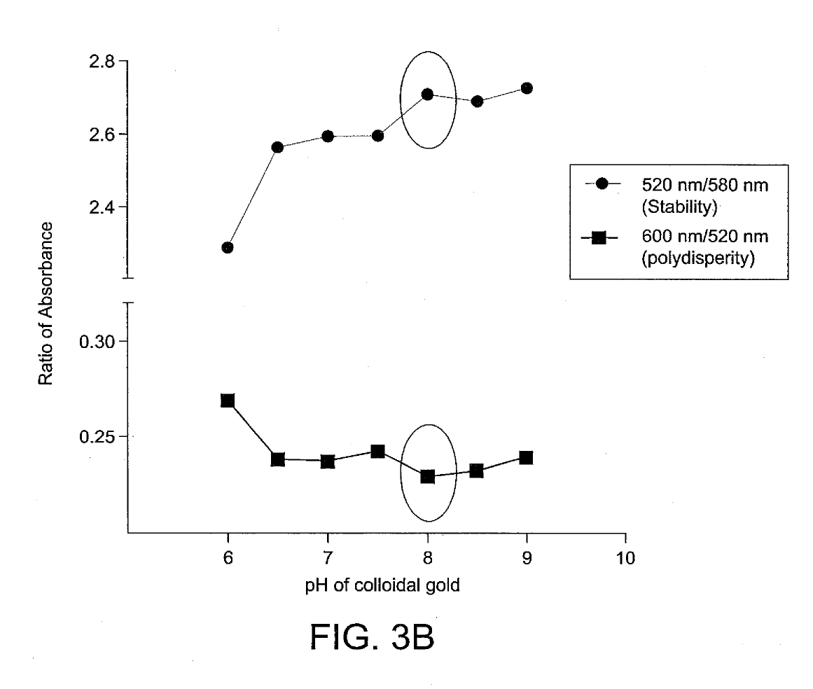

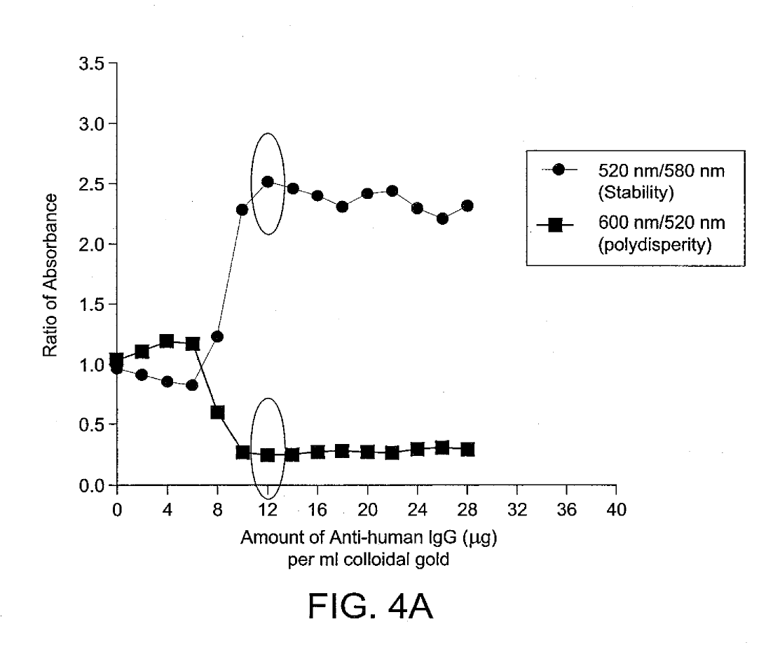

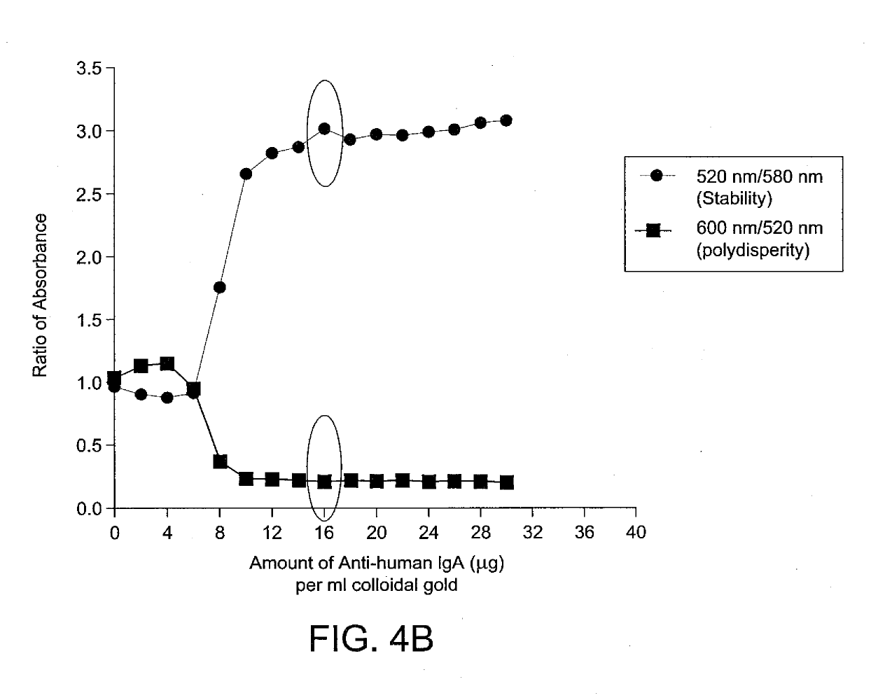

Aggregation testing with different pH values and increasing amounts of goat anti-human IgG or goat anti-human IgA was performed to produce the conjugated gold. After the addition of NaCl, the optical density (OD) was measured at 520 nm, 580 nm and 600 nm. The OD ratio of 520 nm and 580 nm was used to assess stability, and the OD ratio of 600 nm and 520 nm was used to assess polydispersity. The highest stability and lowest polydispersity were found when the colloidal gold was conjugated to both anti-human IgG and IgA at a pH of 8.0, as shown in FIGS. 3A and 3B. A minimum of 12 μg of goat anti-human IgG or 16 μg of goat anti-human IgA were found to be required to stabilize a 1 mL colloidal gold solution, as shown in FIGS. 4A and 4B.

The present test strip 10 was tested for detection of S. Typhi LPS specific IgG in a lymphocyte culture supernatant of 54 blood culture confirmed patients (48 S. Typhi positive and 6 S. Paratyphi A). All S. Typhi and 5 S. Paratyphi A bacteremic patients tested positive according to the present strip test. The assay was also tested in a lymphocyte culture supernatant prepared from venous blood of 88 blood culture negative patients (clinically suspected to have enteric fever), as well as 28 healthy controls and 35 patients with other febrile illness (tuberculosis, kala-azar, dengue and non-Salmonella bacteremia). The assay tested positive for 19 blood culture negative patients and negative for all healthy controls as well as for the all patients with other febrile illness. These results are shown below in Table 3. The test strip detecting S. Typhi MP specific IgA response showed inconsiderable intensity of the control line and test line when it was tested with a sample from a culture of confirmed patients. Further, the test strip detecting MP specific IgG showed high sensitivity for blood culture positive patients but showed cross-reactivity with specimens from other febrile illness.

TABLE 3
Results of Strip Test Detecting LPS-IgG

| | No. of | Strip test | Strip test |
|---|---|---|---|
| Categories of study participants | Individuals | positive | negative |
| Patients with S. Typhi bacteremia | 48 | 48 | 0 |
| Patients with S. Paratyphi A | 6 | 5 | 1 |
| bacteremia | | | |
| Patients who were clinically | 88 | 19 | 69 |
| suspected for enteric fever but | | | |
| blood culture negative | | | |
| Patients with febrile illness other | 35 | 0 | 35 |
| than enteric fever | | | |
| Healthy controls | 28 | 0 | 28 |

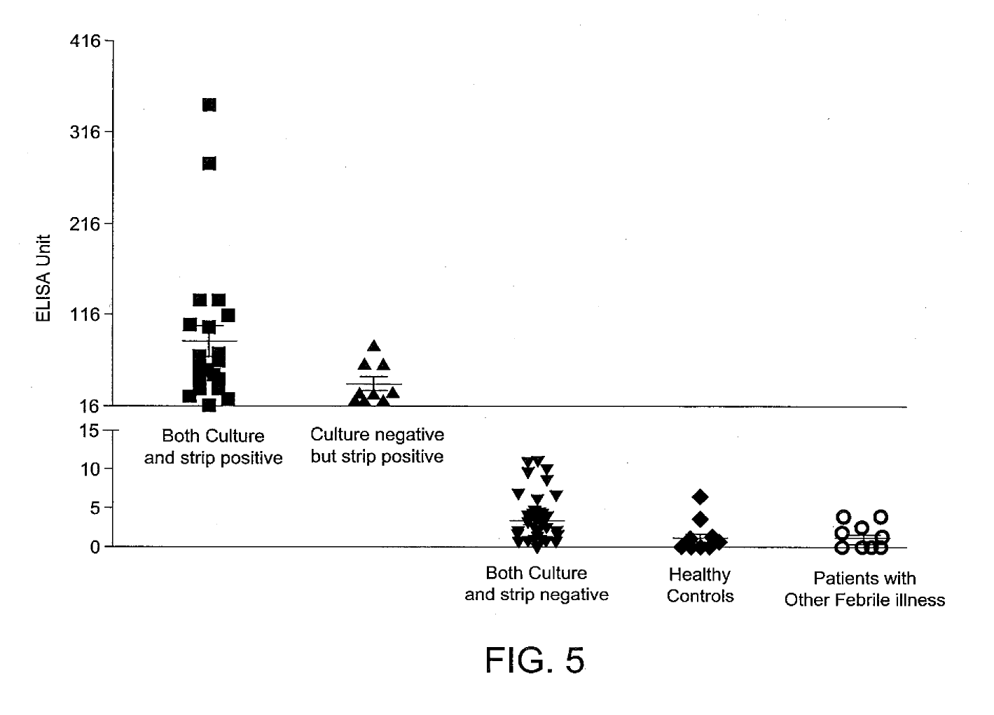

IgG antibody responses were measured using ELISA in a lymphocyte culture supernatant collected from different categories of patients. Among them, 22 patients were positive by blood culture and strip test, 9 patients were negative by blood culture and positive by strip test, and 35 patients were negative for both blood culture and strip test. LPS-IgG responses were also measured in 16 healthy individuals and 16 other febrile illness patients. The patients who had LPS- IgG response of ≧16 EU tested positive by the strip, as illustrated in FIG. 5.

The sensitivity and specificity of the IgG LPS-specific LFD was calculated using OpenEpi, Version 3, an open source calculator for the evaluation of diagnostic test developed at the Rollins School of Public Health of Emory University. The test had a sensitivity of 98% compared against blood culture, and a specificity that ranged from 78-100% (70-100, 95% CI), depending on the definition of a true negative, as shown below in Table 4.

TABLE 4
Sensitivity, Specificity, Positive Predictive Value and Negative
Predictive Value of Strip Test Detecting LPS-IgG

| Test | Sensitivity (%) | Specificity (%) |
|---|---|---|
| Strip test^{a} | 98 (95% CI: 90-99) | 100 (95% CI: 94-100) |
| Strip test^{b} | 98 (95% CI: 90-99) | 87 (95% CI: 81-91) |
| Strip test^{c} | 98 (95% CI: 90-99) | 78 (95% CI: 68-85) |

^{a}Considering blood culture positive patients as positive cases, and healthy individuals and patients with other febrile illnesses as negative cases
^{b}Considering blood culture positive patients as positive cases, and blood culture negative patients, healthy individuals and patients with other febrile illnesses as negative cases
^{c}Considering blood culture positive patients as positive cases, and only blood culture negative patients as negative cases

The present enteric fever test can be performed using 1 mL of venous blood, an erythrocyte lysis buffer, a tabletop centrifuge, and a 37° C. incubator without CO₂. The testing is performed by detecting responses visually with dipstick 10. Although such a test cannot be used at the bedside, results with better precision and reliability can be available after about 24 hours to about 48 hours.

Using the definitions of patient categories given above (i.e., participants whose blood cultures were positive for S. Typhi or S. Paratyphi A were defined as definitive cases of enteric fever), the present invention was found to have a sensitivity of 98%. Further, when participants with other febrile illnesses and healthy controls were defined as definitive negatives, the present invention was found to have a specificity of 100%.

It should be noted that the present invention also detected patients with S. Paratyphi A bactermia. Paratyohoid fever caused by S. Paratyphi A accounts for up to 1 in 5 cases of enteric fever is some areas of Asia, including Bangladesh, and paratyphoid and typhoid fevers can be clinically indistinguishable. S. Typhi LPS serotype is defined by the O antigen, determined by the O-specific oligo and polysaccharides associated with the LPS. S. Typhi O antigens include serotypes 9 and 12, often expressed on the same organism. S. Paratyphi A antigens include serotypes 1, 2 and 12. The identification of S. Paratyphi A infected patients by the dipstick assay presumably rests upon the detection of circulating lymphocytes expressing anti-12 O-antigen antibodies in these individuals. Invasive salmonellosis is a distinct clinical entity from enteric fever in that it is caused by traditionally non-typhoidal strains of Salmonella enterica, especially S. Typhimurium and S. Enteriditis.

Such invasive non-typhoidal salmonellosis (iNTS) is a significant cause of mortality in malnourished and immunocompromised children, especially HIV infected individuals in sub-Saharan Africa. Although the present assay was not assessed in patients with iNTS, it is encouraging to note that both S. Typhimurium and S. Enteriditis can express O antigen 12, suggesting that the present invention could detect at least a subset of individuals with iNTS.

It is to be understood that the present invention is not limited to the embodiments described above, but encompasses any and all embodiments within the scope of the following claims.

We claim:

1. A lateral flow test strip for detecting enteric fever, comprising:
a backing having an elongated chromatographic stationary phase layer disposed thereon;
a test line, of Salmonella enterica serovar Typhi (S. Typhi) antigen extending transversely across the elongated chromatographic stationary phase layer;
a color indicator in the visible light spectrum;
a conjugate pad of absorbent fibers attached to the backing, the conjugate pad having a first mammal's anti-human antibody adsorbed thereon, the first mammal's anti-human antibody being conjugated to the color indicator, the conjugate pad overlapping the elongated chromatographic stationary phase layer; and
a control line of anti-first mammal IgG antibody extending transversely across the elongated chromatographic stationary phase layer, the test line being spaced apart from the control line.

2. The lateral flow test strip for detecting enteric fever according to claim 1, wherein the S. Typhi antigen comprises an antigen derived from S. Typhi strain Ty21a.

3. The lateral flow test strip for detecting enteric fever according to claim 1, wherein the S. Typhi antigen comprises an antigen derived from S. Typhi wild-type strain ST-004.

4. The lateral flow test strip for detecting enteric fever according to claim 1, wherein the first mammal's anti-human antibody comprises goat anti-human IgG.

5. The lateral flow test strip for detecting enteric fever according to claim 4, wherein the anti-first mammal IgG antibody comprises rabbit anti-goat IgG.

6. The lateral flow test strip for detecting enteric fever according to claim 1, wherein the first mammal's anti-human antibody comprises goat anti-human IgA.

7. The lateral flow test strip for detecting enteric fever according to claim 6, wherein the anti-first mammal IgG antibody comprises rabbit anti-goat IgG.

8. The lateral flow test strip for detecting enteric fever according to claim 1, wherein said color indicator comprises colloidal gold nanoparticles exhibiting the color red in the visible light spectrum.

9. The lateral flow test strip for detecting enteric fever according to claim 8, wherein said colloidal gold nanoparticles have an average diameter of approximately 20 nm.

10. The lateral flow test strip for detecting enteric fever according to claim 1, wherein said chromatographic stationary phase layer comprises a nitrocellulose membrane.

11. The lateral flow test strip for detecting enteric fever according to claim 1, wherein the absorbent fibers of said conjugate pad comprise glass fibers.

12. The lateral flow test strip for detecting enteric fever according to claim 1, further comprising an absorbent sink pad secured to the backing, the absorbent sink pad being positioned longitudinally opposite the conjugate pad.

13. The lateral flow test strip for detecting enteric fever according to claim 12, wherein the absorbent sink pad comprises cellulose fibers.

14. A lateral flow test strip for detecting enteric fever, comprising:
a backing having an elongated chromatographic stationary phase layer disposed thereon;
a test line of Salmonella enterica serovar Typhi (S. Typhi) antigen extending transversely across the elongated chromatographic stationary phase layer;
a color indicator in the visible light spectrum, said color indicator including colloidal gold nanoparticles;
a conjugate pad of absorbent fibers attached to the backing, the conjugate pad having a first mammal's anti-human antibody adsorbed thereon, the first mammal's anti-human antibody being conjugated to the color indicator, the conjugate pad overlapping the elongated chromatographic stationary phase layer;
an absorbent sink pad secured to the backing, the absorbent sink pad being positioned longitudinally opposite the conjugate pad; and
a control line of anti-first mammal IgG antibody extending transversely across the elongated chromatographic stationary phase layer, the test line being spaced apart from the control line.

15. The lateral flow test strip for detecting enteric fever according to claim 14, wherein the S. Typhi antigen comprises an antigen derived from S. Typhi strain Ty21a.

16. The lateral flow test strip for detecting enteric fever according to claim 14, wherein the S. Typhi antigen comprises an antigen derived from S. Typhi wild-type strain ST-004.

17. The lateral flow test strip for detecting enteric fever according to claim 14, wherein the first mammal's anti-human antibody comprises goat anti-human IgG.

18. The lateral flow test strip for detecting enteric fever according to claim 17, wherein the anti-first mammal IgG antibody comprises rabbit anti-goat IgG.

19. The lateral flow test strip for detecting enteric fever according to claim 14, wherein the first mammal's anti-human antibody comprises goat anti-human IgA.

20. The lateral flow test strip for detecting enteric fever according to claim 19, wherein the anti-first mammal IgG antibody comprises rabbit anti-goat IgG.
